(19) United States
(12) Patent Application Publication
(10) Pub. No.: US 2021/0330931 A1
(43) Pub. Date: Oct. 28, 2021

(54) PEDIATRIC CATHETER

(71) Applicant: Vanderbilt University, Nashville, TN (US)

(72) Inventors: George T. Nicholson, Nashville, TN (US); Dana R. Janssen, Brentwood, TN (US); Thomas P. Doyle, Brentwood, TN (US)

(21) Appl. No.: 17/273,407

(22) PCT Filed: Sep. 5, 2019
(86) PCT No.: PCT/US2019/049716

§ 371 Date: Mar. 4, 2021

Related U.S. Application Data
(60) Provisional application No. 62/727,373, filed on Sep. 5, 2018.

(51) Int. Cl. A61M 25/00

(52) U.S. Cl. CPC A61M 25/007; A61M 2025/0177; A61M 25/0032; A61M 25/0052

(57) ABSTRACT

A pediatric catheter includes a shaft with a distal end and a proximal end and a first length of the shaft is defined between the distal end and the proximal end. The shaft further includes a first section positioned at the distal end and having an elongate tip. The first section has an outer wall and a first aperture extending therethrough, and at least a portion of the outer wall has a tapered surface. The shaft includes a second section extending from the first section towards the proximal end. The second section has an arcuate wall and a concave wall integrally formed with the arcuate wall. The arcuate wall is coextensive with at least a portion of the outer wall of the first section, and a second aperture extends through the second section. At least a portion of the second aperture is offset from the first aperture

### CROSS-REFERENCE TO RELATED APPLICATIONS

This application is a non-provisional of and claims the benefit of U.S. Provisional Patent Application No. 62/727,373, filed on Sep. 5, 2018, the entire contents of which are incorporated herein by reference.

### FIELD OF THE INVENTION

This disclosure relates to catheters. In particular, this application relates to pediatric angiographic, hemodynamic, and multi-track catheters.

### BACKGROUND

The conventional multi-track angiographic catheter is widely used in pediatric and adult interventional labs across the country. However, there are many downsides to this catheter.

First, conventional multi-track catheters can often cause vessel damage because of its non-tapered tip. The non-tapered tip can lead to serious vessel wall damage, particularly while attempting to cross a region that was recently intervened upon. A vessel wall has three layers: the intima, media, and adventitia. To achieve a successful angioplasty result, one must tear through at least the intima. It is important to note that this tearing is uncontrollable. That is, an interventionist cannot control where along the intima the vessel will tear. Current theory proposes that the intima will tear at its weakest point. In time, as the vessel heals, the expectation is that the vessel will heal at this larger luminal diameter. Therefore, when a physician assesses the damage during a follow-up angiography, a multi-track catheter must be advanced cautiously into position. The abrupt transition of the non-tapered tip of the catheter can result in further, unintended, tearing of a vessel wall.

Second, conventional multi-track catheters can often cause blood loss. The circular catheter shaft that is inserted over a circular guidewire will often lead to malfunction of the sheath's hemostatic valve and result in substantial blood loss during the procedure. This may have a severe detrimental effect in the pediatric population.

Finally, conventional multi-track catheters are difficult to track. Typically, multi-track catheters only have one centimeter at the catheter tip in contact with the guidewire. Such minimal contact with the guidewire often times leads to difficulty in catheter advancement to the desired location. To circumvent this issue, an additional guidewire and/or another catheter needs to be inserted into the patient, both of which increase the cost, may result in the need for a larger vascular entry sheath, and decrease the efficiency of the procedure.

### SUMMARY

In one embodiment, a pediatric catheter includes a shaft with a distal end and a proximal end, a length of the shaft defined between the distal end and the proximal end. The shaft also includes a first section positioned near the distal end, the first section being defined by an outer wall and including a first aperture extending therethrough and a second aperture extending therethrough. The first aperture and the second aperture are at least partially separated by a concave wall that is integrally formed with the outer wall, and at least a portion of the outer wall has a tapered surface. The shaft also includes a second section extending from the first section towards the proximal end. The second section is at least partially defined by a concave wall that is coextensive with the concave wall of the first section, the second section having a third aperture extending therethrough, the aperture being coextensive with the second aperture of the first section. The catheter also has a hub coupled to the proximal end and configured to receive a guidewire therethrough.

In another embodiment, a pediatric catheter includes a shaft with a distal end and a proximal end, a first length of the shaft defined between the distal end and the proximal end. The shaft also includes a first section positioned at the distal end and having an elongate tip. The first section has an outer wall and a first aperture extending therethrough, and at least a portion of the outer wall having a tapered surface. The shaft also includes a second section extending from the first section towards the proximal end. The second section has an arcuate wall and a concave wall integrally formed with the arcuate wall. The arcuate wall is coextensive with at least a portion of the outer wall of the first section, and a second aperture extends through the second section. At least a portion of the second aperture is offset from the first aperture. A hub us coupled to the proximal end and configured to receive a guidewire therethrough.

In a further embodiment, a pediatric catheter includes a shaft with a distal end and a proximal end, a length of the shaft defined between the distal end and the proximal end. The shaft also including a first section positioned near the distal end. The first section is defined by an outer wall and includes a first aperture extending therethrough and a second aperture extending therethrough. At least a portion of the outer wall has a tapered surface. The shaft also includes a second section extending from the first section to the proximal end, and the second section is defined by an outer wall that is coextensive with a portion of the outer wall of the first section. The second section has a third aperture that is coextensive with the second aperture of the first section. A plurality of holes extend through the outer wall of the first section and are configured to allow fluid to flow from the second aperture. The holes are positioned circumferentially about outer wall first section, and each of the holes is positioned at different distances from the distal end. A hub coupled to the proximal end and configured to receive a guidewire therethrough.

Other aspects of the invention will become apparent by consideration of the detailed description and accompanying drawings.

### BRIEF DESCRIPTION OF THE DRAWINGS

FIG. 1 is a top view of a portion of a catheter according to one embodiment.

FIG. 2 is a detailed view of a portion of the catheter shown in FIG. 1.

FIG. 3 is a top view of another portion of the catheter of FIG. 1.

FIG. 4 is a cross-sectional view of a portion of the catheter of FIG. 1 along the line 4-4 of FIG. 3.

FIG. 5 is a cross-sectional view of a portion of the catheter of FIG. 1 along the line 5-5 of FIG. 2.

FIG. 6 is bottom view of a portion of the catheter of FIG. 1.

FIG. 7A is a detailed view of a catheter according to another embodiment.

FIG. 7B is a cross-sectional view along a longitudinal axis of the catheter of FIG. 7A.

FIG. 8A is a side view of a catheter according to another embodiment.

FIG. 8B is a bottom view of the catheter of FIG. 8A.

FIG. 9 is cross-sectional view of a portion of the catheter of FIG. 8A along the longitudinal axis.

FIG. 10 is detailed bottom view of the catheter of FIG. 8A.

FIG. 11 is a cross-sectional view of a portion of the catheter of FIG. 8A along the line 11-11 in FIG. 8A.

FIG. 12 is a cross-sectional view along a longitudinal axis of a catheter according to another embodiment.

FIG. 13 is a cross-sectional view of a portion of the catheter of FIG. 12 along the line 13-13 in FIG. 12.

FIG. 14 is a cross-sectional view of a catheter according to another embodiment.

FIG. 15A shows the results of Experiment 1 using the catheters of FIG. 14.

FIG. 15B shows results of Experiment 1 using a conventional catheter.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

One or more embodiments are described and illustrated in the following description and accompanying drawings. These embodiments are not limited to the specific details provided herein and may be modified in various ways. Furthermore, other embodiments may exist that are not described herein.

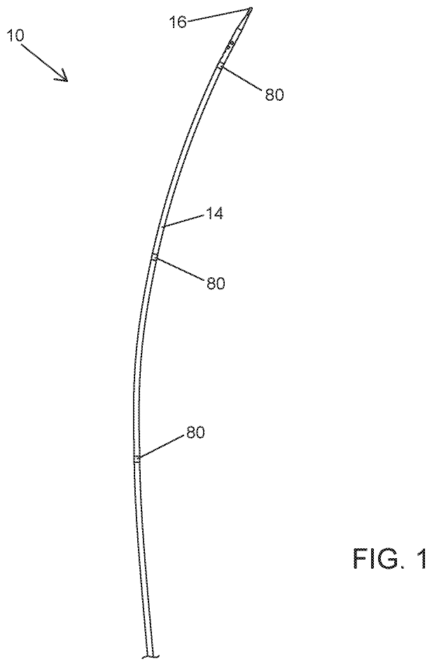

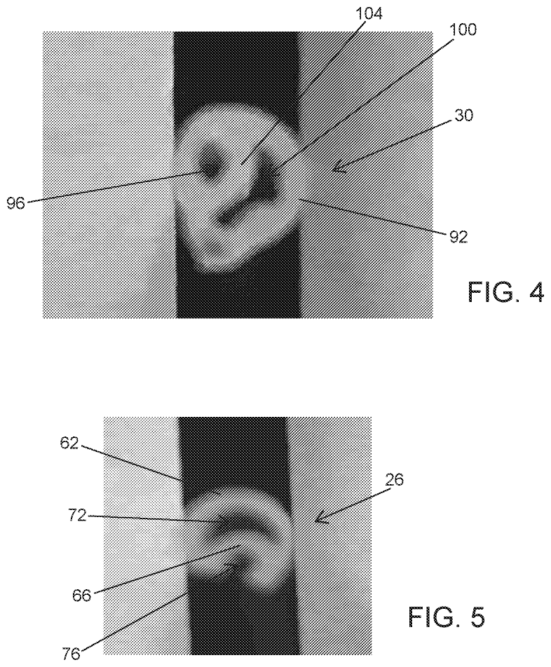

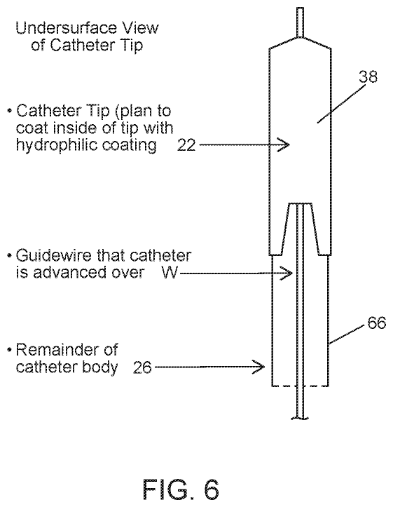

FIGS. 1-7B show a catheter 10 including a shaft 14 that has a distal end 16 and a proximal end 18. A length of the shaft 14 is defined between the distal end 16 and the proximal end 18. The shaft 14 includes a first section 22 positioned near the distal end 16, a second section 26 extending from the first section 22 towards the proximal end 18, a third section 30 extending from the second section 22 to the proximal end 18, and a central or longitudinal axis A. The length of the shaft ranges from 100 mm to 120 mm. The catheter 10 includes radiopaque markers 34 that are spaced along the length of the shaft 14.

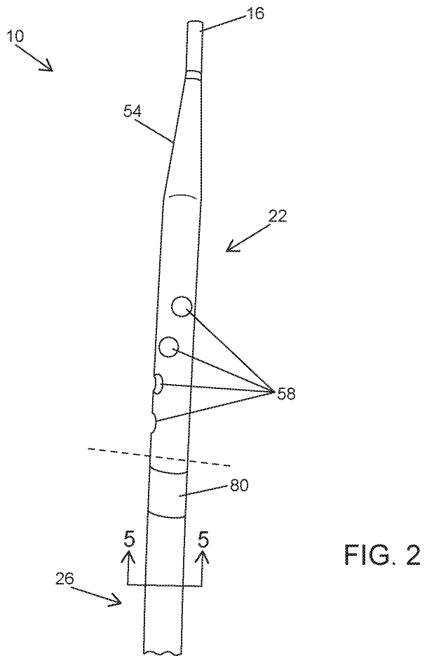

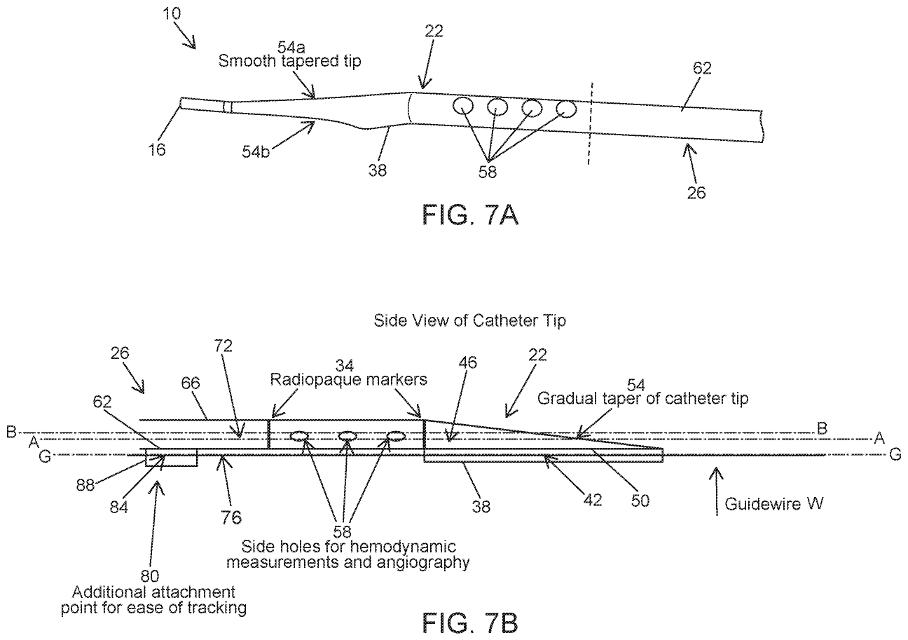

With respect to FIGS. 2, 7A, and 7B, the first section 22 has a first end positioned at the distal end 16 and a second end spaced apart from the first end. The first section 22 has a length that is defined between the first end and the second end. As shown in FIG. 7B, the first section 22 is defined by an outer wall 38 and includes a first aperture 42 extending therethrough and a second aperture 46 extending therethrough. The first aperture 42 and the second aperture 46 are at least partially separated by a concave wall 50 that is integrally formed with the outer wall 38. The first aperture 42 and the second aperture 46 may also be in communication near the distal end 16. Also, at least a portion of the outer wall 38 has a tapered surface 54. A guidewire axis G of the shaft 14 is defined through a center of the first aperture 46. The guidewire axis G is parallel to and offset from the longitudinal axis A. As shown, the outer wall 38 has a surface that tapers from the second end to the first end along the length of the first section 22. A length of the first section 22 ranges from 8 mm to 22 mm. Accordingly, the length of the first section 22 extends along 5% to 25% of the length of the shaft 14. The length of the tapered surface 54 ranges from 10 mm to 20 mm. The tapered surface 54 defines an angle ranging from 15 degrees to 30 degrees relative to the guidewire axis G. The first section 22 may include a hydrophilic coating along the interior of the first aperture 42.

As shown in FIGS. 2, 7A, and 7B, a plurality of holes 58 extend through the outer wall 38 of the first section 22. The holes 58 are positioned along the second aperture 46 in the first section 22 to allow fluid to flow out of the second aperture 46 and into the patient. The holes 58 may be arranged circumferentially about the first section 22 (FIG. 2). Also, each of the holes 58 may be positioned at a different longitudinal distance from the distal end 16, Accordingly, the holes 58 may be offset from one another in a clockwise direction and a horizontal direction (e.g., in a direction that is parallel to the longitudinal axis A). In other embodiments, the holes 58 may be positioned in an array that extends parallel to the longitudinal axis A, as is shown in at least FIGS. 7A and 7B. Also, as shown in FIGS. 7A-7B, the outer wall 38 may have a first tapered surface 54a and second tapered surface 54b, that have different taper angles.

With respect to FIGS. 2, 5, 6 and 7B, the second section 26 includes an arcuate wall 62 and a concave wall 66 that is integrally formed with the arcuate wall 62. The arcuate wall 62 is coextensive with at least a portion of the outer wall 38 of the first section 22, and the concave wall 66 is coextensive with the concave wall 50 of the first section 22, The second section 26 has a third aperture 72 extending therethrough that is also coextensive with the second aperture 46 of the first section 22, The concave wall 66 of the second section creates a recess 76 that is aligned with the first aperture 42. The radius of curvature of the concave wall 66 ranges between 0.40 mm and 0.50 mm. A plurality of reinforcement elements 80 are coupled to and positioned along the second section 26 such that an opening 84 is defined between the concave wall 66 and a surface 88 of each of the reinforcement element 80. In the illustrated embodiment, the reinforcement elements are circular bands. The second section 26 makes up about ¼ to ⅔ of the length of the shaft 14, and the reinforcement elements 80 are spaced about 2 mm to 8 mm away from one another along the second section 26. The reinforcement elements 80 have a length of between 2 mm and 8 mm.

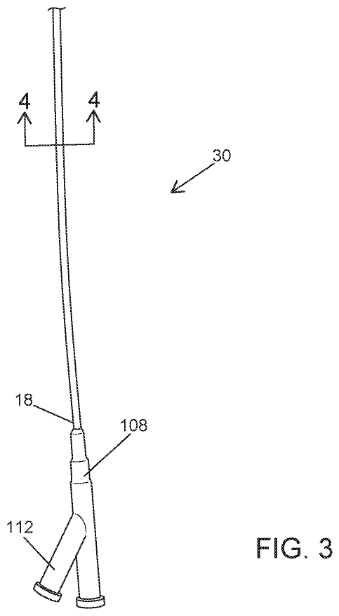

With respect to FIG. 3, the third section 30 has a length that is defined between the second section 26 and the proximal end 18 of the shaft 14. As shown in FIG. 5, the third section 30 is defined by an outer wall 92, and includes a fourth aperture 96 extending therethrough and a fifth aperture 100 extending therethrough. The fourth aperture 96 and the fifth aperture 100 are separated by a concave wall 104 that is integrally formed with the outer wall 92. At least a portion of the outer wall 92 is coextensive with the arcuate wall 62 of the second section 22 and the concave wall 104 is coextensive with the concave wall 66 of the second section 22. The fourth aperture 96 is aligned with the recess 76 of the second section 22 and the fifth aperture 100 is coextensive with the third aperture 72. Accordingly, the second aperture 46 of the first section 22, the third aperture 72 of the second section 22, and the fifth aperture 100 of the third section 30 define a single elongate aperture that extends through the first, second, and third sections 22, 26, 30 along an axis B. The axis B is parallel to both the longitudinal axis A and the guidewire axis B, but is also offset from both the longitudinal axis A and guidewire axis G. The second, third, and fifth apertures 46, 72, 100 (i.e., the single elongate aperture is) are crescent-shaped in the illustrated embodiment. The second, third, and fifth apertures 46, 72, 100 (i.e., the single elongate aperture) guide fluid (e.g., contrast dye) therethrough from the proximal end to the holes 58.

As shown in FIG. 3, a hub or luer 108 is coupled to the proximal end 18 and includes a port 112 in fluid communication with the second, third, and fifth apertures 46, 72, 100 (i.e., the single elongate aperture). The port 112 is configured to receive a hemostatic valve (not shown).

The catheter 10 of FIGS. 1-7B is configured to be guided over a guidewire W. In particular, the first aperture 46, the recess 76, the fifth aperture 100, and the hub 108 are configured to receive and be guided by the guidewire W. As shown in FIG. 1, the guidewire \V is supported by surfaces 88 of the reinforcement members 80 as it is guided through the respective openings 84.

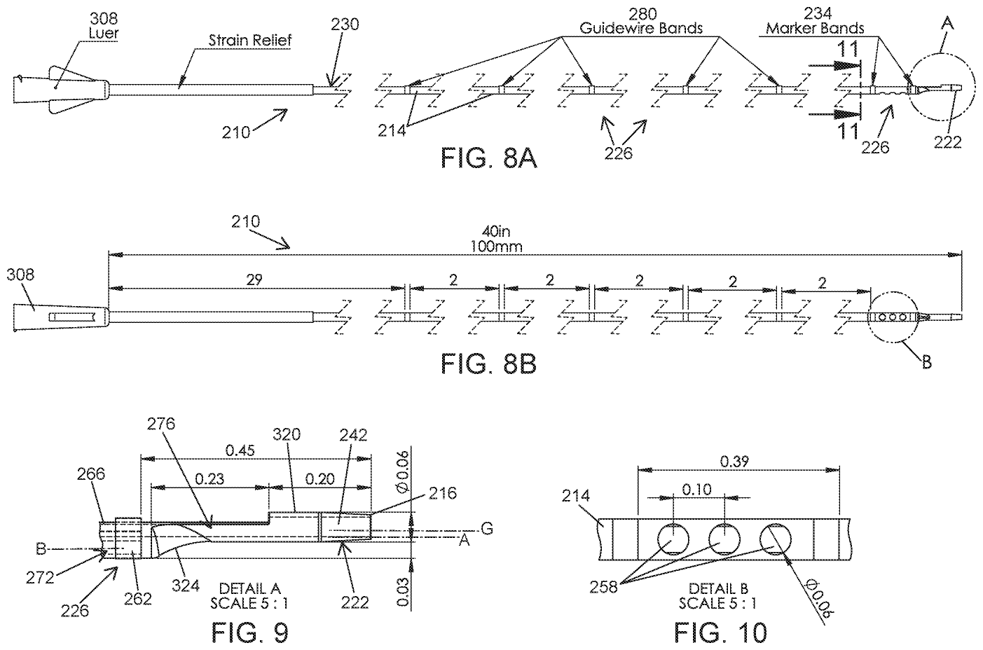

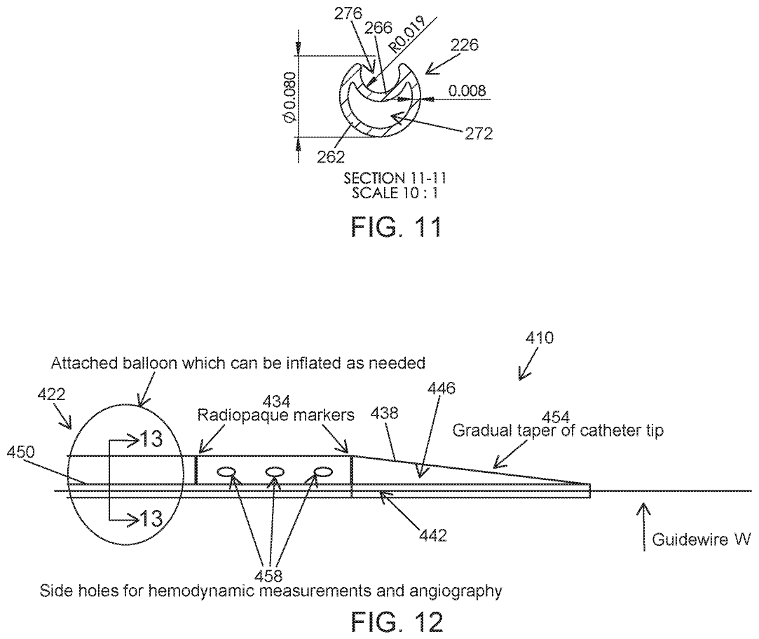

FIGS. 8A-11 illustrate a catheter 210 according to another embodiment. The embodiment of FIGS. 8A-11 is similar to the embodiments of FIGS. 1-7B; therefore, like structure will be identified by like reference numerals plus “200” and only the difference will be discussed hereafter. The first section 222 includes an outer wall 320 that defines the first aperture 242. The outer wall 320 as a tapered surface at the distal end 216 of the shaft 214. The second section 226 of the catheter 210 extends from first section 222. Like the second section 26 of FIGS. 1-7B, the second section 226 includes an arcuate wall 262 that is integrally formed with the concave wall 266. The concave wall 266 is integrally formed with at least a portion of the outer wall 320 of the first section 222 and the arcuate wall 262 is integrally formed with at least a portion of the outer wall 320 of the first section. The first aperture 242 is aligned with the recess 276 created by the concave wall 266 of the second portion 226. At least a portion of the arcuate wall 262 has a tapered surface 324.

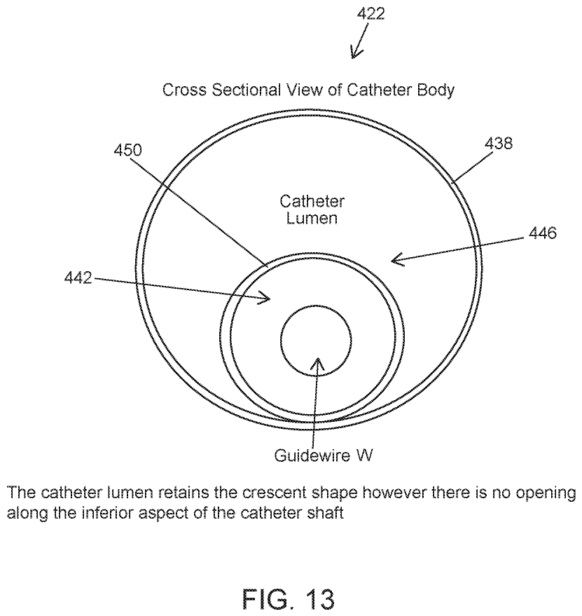

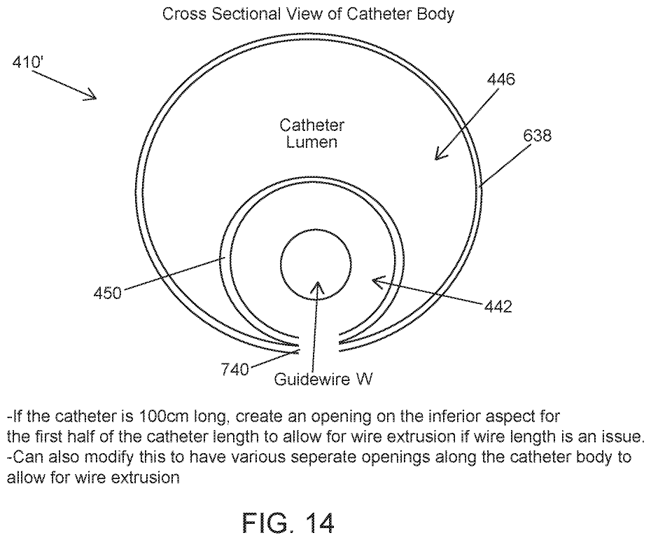

FIGS. 12-13 illustrate a catheter 410 according to another embodiment. The embodiment of FIGS. 12-13 is similar to the embodiments of FIGS. 1-7B; therefore, like structure will be identified by like reference numerals plus “400” and only the difference will be discussed hereafter. The catheter 410 of FIGS. 12-13 is enclosed along the entire length of the shaft 414. Therefore, the catheter 410 has a single, first section 422 that extends between the proximal and distal ends 416, 418. Accordingly, the reinforcement elements 80 of the embodiment of FIGS. 12-13 are not necessary. The catheter 410 of FIGS. 12-13 is configured to be guided over a guidewire W. In particular, the first aperture 442 and the hub (not shown) are configured to receive and be guided by the guidewire W. As shown in FIG. 14, the catheter 410′ may include an opening 740 in at least a portion of the outer wall 438, such that the entire catheter 410′ is not enclosed.

The catheters 10, 210, 410, 410′ shown and described in FIGS. 1-14 are applicable for both pediatric and adult cardiac uses for both diagnostic and interventional cases. The advantages of this design are many, some of which are listed herein. First, the crescent shaped catheter shafts 14, 214, 414 allow for the hemostatic valve to maintain better function. Second, the catheters 10, 210, 410, 410′ are compatible with a smaller sheath size, which is extremely important in the pediatric age group. Third, the longer catheter tip improves tracking over a guidewire. Fourth, the taper of the distal ends 16, 216, 416, lessens the risk of vessel wall injury. Fifth, the catheters 10, 210, 410, 410′ reduce blood loss. Sixth, the catheter 10, 210, 410, 410′ reduce voids between the hemostatic valve and the catheter. Seventh, is not subjected to deformational change with injection pressures up to 1200 psi (which is twice the normal injection pressure used in routine angiography). Finally, the hydrophilic coating along the interior of the catheter tip improves ease of multi-track catheter delivery and positioning.

### Example 1

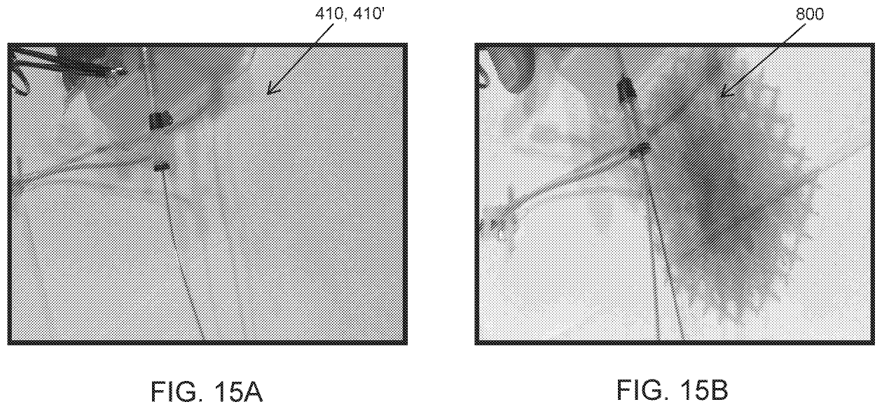

The catheters 410, 410′ of FIGS. 12-13 and 14 were compared to a conventional catheter 800 in this Example. A sheath (i.e., a 8 Fr Terumo sheath) was inserted into a one liter bag of normal saline. A guidewire was then inserted through the sheath. Through the same sheath, one at a time, each of six catheters were inserted into the bag over the guidewire. At a simulated arterial blood pressure of 70/30 mmHg, the volume of fluid loss was recorded over a two minute testing time at a heart rate of 60 beats per minute (“bpm). As shown in Table 1 below, the catheters 410, 410′ of FIGS. 12-13 (e.g., Prototype #1) and FIG. 14 (e.g., Prototype #2) resulted in less blood loss than the conventional catheters (e.g., the rows designated “Multi Track”). Exemplary pictures (showing dyed saline) of the experiment are shown in FIGS. 15A and 15B. As shown in FIG. 15A, the catheters 410, 410′ of FIGS. 12-14 do not lose hemostatic valve integrity, which resulted in no fluid loss. However, the conventional catheters 800 (FIG. 15B) did show loss of hemostatic valve integrity and therefore fluid loss.

| Catheter | French Size | Wire OD (″) | Volume Lost (cc) |
|---|---|---|---|
| Prototype #1 | 6 | 0.025 | 0.5 |
| Prototype #2 | 6 | 0.025 | 0.4 |
| Multi Track | 4 | 0.025 | 7 |
| Multi Track | 4 | 0.035 | 13 |
| Multi Track | 5 | 0.035 | 15 |
| Multi Track | 6 | 0.035 | 22 |

For reasons of completeness, various aspects of the invention are set out in the following numbered clauses:

Clause 1. A pediatric catheter comprising:

a shaft including a distal end and a proximal end, a length of the shaft defined between the distal end and the proximal end, the shaft further including:

a first section positioned near the distal end, the first section being defined by an outer wall and including a first aperture extending therethrough and a second aperture extending therethrough, the first aperture and the second aperture being at least partially separated by a concave wall that is integrally formed with the outer wall, at least a portion of the outer wall having a tapered surface; and

a second section extending from the first section towards the proximal end, the second section being at least partially defined by a concave wall that is coextensive with the concave wall of the first section, the second section having a third aperture extending therethrough, the aperture being coextensive with the second aperture of the first section;

a hub coupled to the proximal end and configured to receive a guidewire therethrough.

Clause 2. The catheter of claim 1, wherein the tapered surface defines an angle ranging from 15 degrees to 30 degrees relative to an axis that is defined through the first aperture.

Clause 3. The catheter of claim 1, wherein a length of the tapered surface ranges from 10 mm to 20 mm.

Clause 4. The catheter of claim 1, wherein the first section extends along about 5% to 20% of the length of the shaft.

Clause 5. The catheter of claim 1, further comprising a plurality of reinforcement elements coupled to the second section, an opening being defined between the concave wall and a surface of each of reinforcement element.

Clause 6. The catheter of claim 1, wherein the concave wall of the second section creates a recess that is aligned with the first aperture such that the first aperture and the recess are configured to receive the guidewire.

Clause 7. A pediatric catheter comprising:

a shaft including a distal end and a proximal end, a first length of the shaft defined between the distal end and the proximal end, the shaft further including:

a first section positioned at the distal end and having an elongate tip, the first section having an outer wall and a first aperture extending therethrough, at least a portion of the outer wall having a tapered surface;

a second section extending from the first section towards the proximal end, the second section having an arcuate wall and a concave wall integrally formed with the arcuate wall, the arcuate wall being coextensive with at least a portion of the outer wall of the first section, a second aperture extending through the second section, least a portion of the second aperture being offset from the first aperture;

a hub coupled to the proximal end and configured to receive a guidewire therethrough.

Clause 8. The catheter of claim 7, wherein the tapered surface defines an angle ranging from 15 degrees to 30 degrees relative to an axis that is defined through the first aperture.

Clause 9. The catheter of claim 7, wherein a length of the tapered surface ranges from 10 mm to 22 mm.

Clause 10. The catheter of claim 7, wherein the first section extends along about 5% to 22% of the length of the shaft.

Clause 11. The catheter of claim 7, further comprising a plurality of reinforcement elements coupled to the second section, an opening being defined between the concave wall and a surface of each of reinforcement element.

Clause 12. The catheter of claim 7, wherein the concave wall of the second section creates a recess that is aligned with the first aperture such that the first aperture and the recess are configured to receive the guidewire.

Clause 13. The catheter of claim 7, wherein the second aperture is substantially crescent-shaped.

Clause 14. A pediatric catheter comprising:

a shaft including a distal end and a proximal end, a length of the shaft defined between the distal end and the proximal end, the shaft further including:

a first section positioned near the distal end, the first section being defined by an outer wall and including a first aperture extending therethrough and a second aperture extending therethrough, at least a portion of the outer wall having a tapered surface; and

a second section extending from the first section to the proximal end, the second section being defined by an outer wall that is coextensive with a portion of the outer wall of the first section, the second section having a third aperture that is coextensive with the second aperture of the first section;

a plurality of holes extending through the outer wall of the first section and configured to allow fluid to flow from the second aperture, the holes being positioned circumferentially about outer wall first section, each of the holes being positioned at different distances from the distal end;

a hub coupled to the proximal end and configured to receive a guidewire therethrough.

Clause 15. The catheter of claim 14, wherein the holes are offset from one another in a clockwise direction and in a horizontal direction.

Clause 16. The catheter of claim 14, wherein the second aperture and the third aperture define a crescent-shaped lumen.

Clause 17. The catheter of claim 14, wherein the tapered surface defines an angle ranging from 15 degrees to 30 degrees relative to an axis that is defined through the first aperture.

Clause 18. The catheter of claim 14, wherein a length of the tapered surface ranges from 10 mm to 22 mm.

Clause 19. The catheter of claim 14, wherein the first section extends along about 5% to 22% of the length of the shaft.

Clause 20. The catheter of claim 14, further comprising a plurality of reinforcement elements coupled to the second section, an opening being defined between the concave wall and a surface of each of reinforcement element.

It is understood that the foregoing detailed description is merely illustrative and is not to be taken as limitations upon the scope of the invention, which is defined solely by the appended claims and their equivalents. Various changes and modifications to the disclosed embodiments will be apparent to those skilled in the art. Various features and advantages of the invention are set forth in the following claims.

What is claimed is:

1. A pediatric catheter comprising:
a shaft including a distal end and a proximal end, a length of the shaft defined between the distal end and the proximal end, the shaft further including:
a first section positioned near the distal end, the first section being defined by an outer wall and including a first aperture extending therethrough and a second aperture extending therethrough, the first aperture and the second aperture being at least partially separated by a concave wall that is integrally formed with the outer wall, at least a portion of the outer wall having a tapered surface; and
a second section extending from the first section towards the proximal end, the second section being at least partially defined by a concave wall that is coextensive with the concave wall of the first section, the second section having a third aperture extending therethrough, the aperture being coextensive with the second aperture of the first section;
a hub coupled to the proximal end and configured to receive a guidewire therethrough.

2. The catheter of claim 1, wherein the tapered surface defines an angle ranging from 15 degrees to 30 degrees relative to an axis that is defined through the first aperture.

3. The catheter of claim 1, wherein a length of the tapered surface ranges from 10 mm to 20 mm.

4. The catheter of claim 1, wherein the first section extends along about 5% to 20% of the length of the shaft.

5. The catheter of claim 1, further comprising a plurality of reinforcement elements coupled to the second section, an opening being defined between the concave wall and a surface of each of reinforcement element.

6. The catheter of claim 1, wherein the concave wall of the second section creates a recess that is aligned with the first aperture such that the first aperture and the recess are configured to receive the guidewire.

7. A pediatric catheter comprising:
a shaft including a distal end and a proximal end, a first length of the shaft defined between the distal end and the proximal end, the shaft further including:
a first section positioned at the distal end and having an elongate tip, the first section having an outer wall and a first aperture extending therethrough, at least a portion of the outer wall having a tapered surface;
a second section extending from the first section towards the proximal end, the second section having an arcuate wall and a concave wall integrally formed with the arcuate wall, the arcuate wall being coextensive with at least a portion of the outer wall of the first section, a second aperture extending through the second section, at least a portion of the second aperture being offset from the first aperture;
a hub coupled to the proximal end and configured to receive a guidewire therethrough.

8. The catheter of claim 7, wherein the tapered surface defines an angle ranging from 15 degrees to 30 degrees relative to an axis that is defined through the first aperture.

9. The catheter of claim 7, wherein a length of the tapered surface ranges from 10 mm to 22 mm.

10. The catheter of claim 7, wherein the first section extends along about 5% to 22% of the length of the shaft.

11. The catheter of claim 7, further comprising a plurality of reinforcement elements coupled to the second section, an opening being defined between the concave wall and a surface of each of reinforcement element.

12. The catheter of claim 7, wherein the concave wall of the second section creates a recess that is aligned with the first aperture such that the first aperture and the recess are configured to receive the guidewire.

13. The catheter of claim 7, wherein the second aperture is substantially crescent-shaped.

14. A pediatric catheter comprising:
a shaft including a distal end and a proximal end, a length of the shaft defined between the distal end and the proximal end, the shaft further including:
a first section positioned near the distal end, the first section being defined by an outer wall and including a first aperture extending therethrough and a second aperture extending therethrough, at least a portion of the outer wall having a tapered surface; and
a second section extending from the first section to the proximal end, the second section being defined by an outer wall that is coextensive with a portion of the outer wall of the first section, the second section having a third aperture that is coextensive with the second aperture of the first section;
a plurality of holes extending through the outer wall of the first section and configured to allow fluid to flow from the second aperture, the holes being positioned circumferentially about outer wall first section, each of the holes being positioned at different distances from the distal end;
a hub coupled to the proximal end and configured to receive a guidewire therethrough.

15. The catheter of claim 14, wherein the holes are offset from one another in a clockwise direction and in a horizontal direction.

16. The catheter of claim 14, wherein the second aperture and the third aperture define a crescent-shaped lumen.

17. The catheter of claim 14, wherein the tapered surface defines an angle ranging from 15 degrees to 30 degrees relative to an axis that is defined through the first aperture.

18. The catheter of claim 14, wherein a length of the tapered surface ranges from 10 mm to mm.

19. The catheter of claim 14, wherein the first section extends along about 5% to 22% of the length of the shaft.

20. The catheter of claim 14, further comprising a plurality of reinforcement elements coupled to the second section, an opening being defined between the concave wall and a surface of each of reinforcement element.
